(19) United States
(12) Patent Application Publication
(10) Pub. No.: US 2026/0215919 A1
(43) Pub. Date: Jul. 30, 2026

(54) Prosthetic Heart Valve Dilatation Apparatus

(71) Applicant: Medtronic, Inc., Minneapolis, MN (US)

(72) Inventor: Ryan P. STUBLASKI, Irvine, CA (US)

(21) Appl. No.: 19/147,349

(22) PCT Filed: Jan. 18, 2024
(86) PCT No.: PCT/US2024/011982

§ 371 Date: Jul. 11, 2025

Related U.S. Application Data
(60) Provisional application No. 63/441,513, filed on Jan. 27, 2023.

(51) Int. Cl. A61F 2/24; A61M 25/10

(52) U.S. Cl. CPC A61F 2/2433; A61F 2/2418; A61M 25/1011; A61F 2230/0006; A61F 2250/0039; A61M 2025/1043; A61M 2210/125

(57) ABSTRACT

A prosthetic heart valve assembly includes an annular frame including a first section, a second section, and a waist section between the first section and the second section. An inflation apparatus includes a first balloon for positioning in the first section. The first balloon includes a first balloon diameter in the inflated state such that the first balloon is configured to be in contact with the first section. The inflation apparatus includes a second balloon for positioning in the second section. The second balloon includes a second balloon diameter in the inflated state such that the second balloon is configured to be in contact with the second section. A fluid source is in fluid communication with a catheter to deliver a fluid to the first balloon and to the second balloon. Methods of positioning a heart valve prosthesis and employing an inflation apparatus are provided.

### CROSS-REFERENCE TO RELATED APPLICATIONS

This application claims the benefit of U.S. Provisional Application No. 63/441,513, filed Jan. 27, 2023, the entire contents of which are incorporated herein by reference.

### FIELD

The present disclosure relates generally to a prosthetic heart valve assembly and, more particularly, to an inflation apparatus for positioning within a heart valve prosthesis.

### BACKGROUND

It is known to provide a prosthetic heart valve assembly for implanting a heart valve prosthesis within a target site of the vasculature of a patient. The heart valve prosthesis can be moved from a radially-contracted position to a radially-expanded position. However, expansion of the heart valve prosthesis can be difficult.

### SUMMARY

The following presents a simplified summary of the disclosure to provide a basic understanding of some aspects described in the detailed description.

In aspects, an inflation apparatus is provided for dilating a prosthetic heart valve comprising an annular frame extending along a longitudinal axis between an inflow end and an outflow end. The frame comprises a plurality of struts and is configured to be adjustable between a radially-collapsed position and a radially-expanded position. The frame comprises a first section at the inflow end, a second section at the outflow end, and a waist section positioned between the first section and the second section. A valve prosthesis comprises at least one leaflet attached to the plurality of struts. The inflation apparatus is configured to be positioned within the frame. The inflation apparatus comprises a first balloon configured to be positioned in the first section. The first balloon is configured to be pressurized from a deflated state to an inflated state. The first balloon comprising a first balloon diameter in the inflated state such that the first balloon is configured to be in contact with an interior surface of the first section. A second balloon is configured to be positioned in the second section. The second balloon is configured to be pressurized from a deflated state to an inflated state. The second balloon comprises a second balloon diameter in the inflated state such that the second balloon is configured to be in contact with an interior surface of the second section. A catheter is configured to extend through the waist section between the first balloon and the second balloon. The catheter comprises a first hollow chamber in fluid communication with the first balloon and a second hollow chamber in fluid communication with the second balloon.

In aspects, the first balloon is spaced a separating distance from the second balloon and defines a gap between the first balloon and the second balloon. The catheter extends within the gap.

In aspects, the first balloon is configured to be spaced a first distance from the at least one leaflet and the second balloon is configured to be spaced a second distance from the at least one leaflet.

In aspects, the first balloon diameter is substantially equal to a maximum first diameter of the first section.

In aspects, the second balloon diameter is substantially equal to a maximum second diameter of the second section.

In aspects, the catheter comprises a first shaft portion positioned within the first balloon. The first shaft portion comprises a first opening defining a first fluid passageway between the first hollow chamber and a first interior chamber of the first balloon.

In aspects, the catheter comprises a second shaft portion positioned within the second balloon. The second shaft portion comprises a second opening defining a second fluid passageway between the second hollow chamber and a second interior chamber of the second balloon.

In aspects, an inflation apparatus is provided for dilating a prosthetic heart valve comprising an annular frame extending along a longitudinal axis between an inflow end and an outflow end. The frame comprises a plurality of struts and is configured to be adjustable between a radially-collapsed position and a radially-expanded position. The frame comprises a first section at the inflow end, a second section at the outflow end, and a waist section positioned between the first section and the second section. A valve prosthesis comprises at least one leaflet attached to the plurality of struts. The inflation apparatus is configured to be positioned within the frame. The inflation apparatus comprises a first balloon configured to be positioned in the first section. The first balloon is configured to be pressurized from a deflated state to an inflated state. The first balloon comprises a first balloon diameter in the inflated state such that the first balloon is configured to be in contact with an interior surface of the first section. The first balloon extends between a first balloon end and a second balloon end. The first balloon end is configured to be spaced a first distance from the at least one leaflet and the second balloon end is configured to be spaced a second distance from the at least one leaflet. A catheter is configured to extend through the interior of the frame and comprises a first hollow chamber in fluid communication with the first balloon. A fluid source is in fluid communication with the catheter. The fluid source is configured to deliver a fluid through the first hollow chamber to the first balloon.

In aspects, the inflation apparatus further comprises a second balloon configured to be positioned in the second section. The second balloon is configured to be pressurized from a deflated state to an inflated state. The second balloon comprises a second balloon diameter in the inflated state such that the second balloon is configured to be in contact with an interior surface of the second section. A central balloon is configured to be positioned in the waist section and is attached at one end to the first balloon and at an opposing end to the second balloon. The first balloon, the second balloon, and the central balloon define a continuous interior chamber.

In aspects, the central balloon comprises a central balloon diameter that is less than the first balloon diameter and the second balloon diameter.

In aspects, the catheter extends through the first balloon, the second balloon, and the central balloon. The catheter comprises one or more openings defining a fluid passageway between the first hollow chamber and the interior chamber.

In aspects, methods of dilating a heart valve prosthesis positioned at a treatment site within a patient are provided. The heart valve prosthesis comprises a first section at an inflow end of the heart valve prosthesis, a second section at an outflow end of the heart valve prosthesis, and a waist section positioned between the first section and the second section. The heart valve prosthesis is positioned such that the first section is within an annulus at the treatment site. Methods comprise positioning an inflation apparatus within a central lumen of the heart valve prosthesis. The inflation apparatus comprises a first balloon positioned in the first section, a second balloon positioned in the second section, and a catheter extending between the first balloon and the second balloon. Methods comprise inflating the first balloon such that the first balloon contacts an interior surface of the first section to increase a diameter of the first section. Methods comprise inflating the second balloon such that the second balloon contacts an interior surface of the second section to increase a diameter of the second section.

In aspects, methods comprise, prior to positioning the inflation apparatus, determining that at least one of the first section is radially expanded to a first initial diameter that is less than a first desired diameter or the second section is radially expanded to a second initial diameter that is less than a second desired diameter.

In aspects, inflating the first balloon comprises contacting the interior surface of the first section to radially expand the first section from the first initial diameter to the first desired diameter.

In aspects, inflating the second balloon comprises contacting the interior surface of the second section to radially expand the second section from the second initial diameter to the second desired diameter.

In aspects, inflating the first balloon comprises delivering a first fluid through a first hollow chamber of the catheter and inflating the second balloon comprises delivering a second fluid through a second hollow chamber of the catheter. The first hollow chamber is isolated from the second hollow chamber.

In aspects, inflating the second balloon occurs prior to inflating the first balloon.

In aspects, positioning the inflation apparatus within the central lumen comprises aligning a balloon radiopaque marker of the inflation apparatus with a valve radiopaque marker of the valve prosthesis.

In aspects, aligning the balloon radiopaque marker occurs prior to one or more of inflating the first balloon or inflating the second balloon.

In aspects, the heart valve prosthesis comprises at least one leaflet positioned within the central lumen. The first balloon and the second balloon are spaced apart, and not in contact with, the at least one leaflet after the positioning the inflation apparatus within the central lumen of the heart valve prosthesis.

Additional features and advantages of the aspects disclosed herein will be set forth in the detailed description that follows, and in part will be clear to those skilled in the art from that description or recognized by practicing the aspects described herein, including the detailed description which follows, the claims, as well as the appended drawings. It is to be understood that both the foregoing general description and the following detailed description present aspects intended to provide an overview or framework for understanding the nature and character of the aspects disclosed herein. The accompanying drawings are included to provide further understanding and are incorporated into and constitute a part of this specification. The drawings illustrate various aspects of the disclosure, and together with the description explain the principles and operations thereof.

### BRIEF DESCRIPTION OF THE DRAWINGS

These and other features, aspects and advantages are better understood when the following detailed description is read with reference to the accompanying drawings, in which:

FIG. 1 schematically illustrates example aspects of a transcatheter heart valve prosthesis in accordance with aspects of the disclosure;

FIG. 2 illustrates a top-down view of the transcatheter heart valve prosthesis in accordance with aspects of the disclosure;

FIG. 3 illustrates a side view of a delivery assembly for delivering the transcatheter heart valve prosthesis in accordance with aspects of the disclosure;

FIG. 4 illustrates a side view of the delivery assembly for delivering the transcatheter heart valve prosthesis in accordance with aspects of the disclosure;

FIG. 5 illustrates an introducer sheath in accordance with aspects of the disclosure;

FIG. 6 illustrates an introducer sheath in accordance with aspects of the disclosure;

FIG. 7 schematically illustrates a side view of a transcatheter heart valve prosthesis positioned at a treatment site in accordance with aspects of the disclosure;

FIG. 8 illustrates a perspective view of an inflation apparatus in accordance with aspects of the disclosure;

FIG. 9 illustrates a cross-sectional view of a catheter as viewed along lines 9-9 of FIG. 8 in accordance with aspects of the disclosure;

FIG. 10 illustrates a cross-sectional view of the inflation apparatus as viewed along lines 10-10 of FIG. 8 in accordance with aspects of the disclosure;

FIG. 11 illustrates a side view of the transcatheter heart valve prosthesis positioned at the treatment site in accordance with aspects of the disclosure;

FIG. 12 illustrates a side view of the inflation apparatus positioned within the transcatheter heart valve prosthesis in accordance with aspects of the disclosure;

FIG. 13 illustrates a side view of the inflation apparatus positioned within the transcatheter heart valve prosthesis in accordance with aspects of the disclosure;

FIG. 14 illustrates additional aspects of an inflation apparatus in accordance with aspects of the disclosure; and

FIG. 15 illustrates the inflation apparatus of FIG. 14 positioned within the transcatheter heart valve prosthesis in accordance with aspects of the disclosure.

### DETAILED DESCRIPTION

Aspects will now be described more fully hereinafter with reference to the accompanying drawings in which example aspects are shown. Whenever possible, the same reference numerals are used throughout the drawings to refer to the same or like parts. However, this disclosure may be embodied in many different forms and should not be construed as limited to the aspects set forth herein.

As used herein, the term “about” means that amounts, sizes, formulations, parameters, and other quantities and characteristics are not, and need not be, exact, but may be approximate and/or larger or smaller, as desired, reflecting tolerances, conversion factors, rounding off, measurement error and the like, and other factors known to those of skill in the art.

Ranges can be expressed herein as from “about” one value, and/or to “about” another value. When such a range is expressed, aspects include from the one value to the other value. Similarly, when values are expressed as approximations by use of the antecedent “about,” it will be understood that the value forms another aspect. It will be further understood that the endpoints of each of the ranges are significant both in relation to the other endpoint, and independently of the other endpoint.

Directional terms as used herein—for example up, down, right, left, front, back, top, bottom, upper, lower, etc.—are made only with reference to the figures as drawn and are not intended to imply absolute orientation.

Unless otherwise expressly stated, it is in no way intended that any methods set forth herein be construed as requiring that its steps be performed in a specific order, nor that with any apparatus, specific orientations be required. Accordingly, where a method claim does not actually recite an order to be followed by its steps, or that any apparatus claim does not actually recite an order or orientation to individual components, or it is not otherwise specifically stated in the claims or description that the steps are to be limited to a specific order, or that a specific order or orientation to components of an apparatus is not recited, it is in no way intended that an order or orientation be inferred in any respect. This holds for any possible non-express basis for interpretation, including matters of logic relative to arrangement of steps, operational flow, order of components, or orientation of components; plain meaning derived from grammatical organization or punctuation, and; the number or type of aspects described in the specification.

As used herein, the singular forms “a,” “an,” and “the” include plural references unless the context clearly dictates otherwise. Thus, for example, reference to “a” component includes aspects having two or more such components, unless the context clearly indicates otherwise.

The word “exemplary,” “example,” or various forms thereof are used herein to mean serving as an example, instance, or illustration. Any aspect or design described herein as “exemplary” or as an “example” should not be construed as preferred or advantageous over other aspects or designs. Furthermore, examples are provided solely for purposes of clarity and understanding and are not meant to limit or restrict the disclosed subject matter or relevant portions of this disclosure in any manner. It can be appreciated that a myriad of additional or alternate examples of varying scope could have been presented but have been omitted for purposes of brevity.

As used herein, the terms “comprising,” “including,” and variations thereof shall be construed as synonymous and open-ended, unless otherwise indicated. A list of elements following the transitional phrases comprising or including is a non-exclusive list, such that elements in addition to those specifically recited in the list may also be present.

The terms “substantial,” “substantially,” and variations thereof as used herein are intended to represent that a described feature is equal or approximately equal to a value or description. For example, a “substantially planar” surface is intended to denote a surface that is planar or approximately planar. Moreover, “substantially” is intended to denote that two values are equal or approximately equal. The term “substantially” may denote values within about 10% of each other, for example, within about 5% of each other, or within about 2% of each other.

Modifications may be made to the instant disclosure without departing from the scope or spirit of the claimed subject matter. Unless specified otherwise, “first,” “second,” or the like are not intended to imply a temporal aspect, a spatial aspect, an ordering, etc. Rather, such terms are merely used as identifiers, names, etc. for features, elements, items, etc. For example, a first end and a second end generally correspond to end A and end B or two different ends.

Unless otherwise indicated, the terms “distal” and “proximal” are used in the following description with respect to a position or direction relative to the treating clinician. “Distal” and “distally” are positions distant from or in a direction away from the clinician, and “proximal” and “proximally” are positions near or in a direction toward the clinician. In addition, the term “self-expanding” may be used in the following description with reference to one or more valve or stent structures of the prostheses hereof and is intended to convey that the structures are shaped or formed from a material that can be provided with a mechanical memory to return the structure from a compressed or constricted delivery configuration to an expanded deployed configuration or vice versa. Non-exhaustive exemplary self-expanding materials include stainless steel, a pseudo-elastic metal such as a nickel titanium alloy or nitinol, various polymers, or a so-called super alloy, which may have a base metal of nickel, cobalt, chromium, or other metal. Mechanical memory may be imparted to a wire or stent structure by thermal treatment to achieve a spring temper in stainless steel, for example, or to set a shape memory in a susceptible metal alloy, such as nitinol. Various polymers that can be made to have shape memory characteristics may also be suitable for use in aspects hereof to include polymers such as polynorborene, trans-polyisoprene, styrene-butadiene, and polyurethane. As well poly L-D lactic copolymer, oligo caprylactone copolymer and poly cyclo-octine can be used separately or in conjunction with other shape memory polymers.

Diseases associated with heart valves, such as those caused by damage or a defect, can include stenosis and valvular insufficiency or regurgitation. For example, valvular stenosis causes the valve to become narrowed and hardened which can prevent blood flow to a downstream heart chamber from occurring at the proper flow rate and may cause the heart to work harder to pump the blood through the diseased valve. Valvular insufficiency or regurgitation occurs when the valve does not close completely, allowing blood to flow backwards, thereby causing the heart to be less efficient. A diseased or damaged valve, which can be congenital, age-related, drug-induced, or in some instances, caused by infection, can result in an enlarged, thickened heart that loses elasticity and efficiency. Some symptoms of heart valve diseases can include weakness, shortness of breath, dizziness, fainting, palpitations, anemia and edema, and blood clots which can increase the likelihood of stroke or pulmonary embolism. Symptoms can often be severe enough to be debilitating and/or life threatening.

Heart valve prostheses have been developed for repair and replacement of diseased and/or damaged heart valves. Such heart valve prostheses can be percutaneously delivered and deployed at the site of the diseased heart valve through catheter-based delivery systems. Such heart valve prostheses generally include a frame or stent and a prosthetic valve mounted within the frame. Such heart valve prostheses are delivered in a radially compressed or crimped configuration so that the heart valve prosthesis can be advanced through the patient's vasculature. Once positioned at the treatment site, the heart valve prosthesis is expanded to engage tissue at the diseased heart valve region to, for instance, hold the heart valve prosthesis in position.

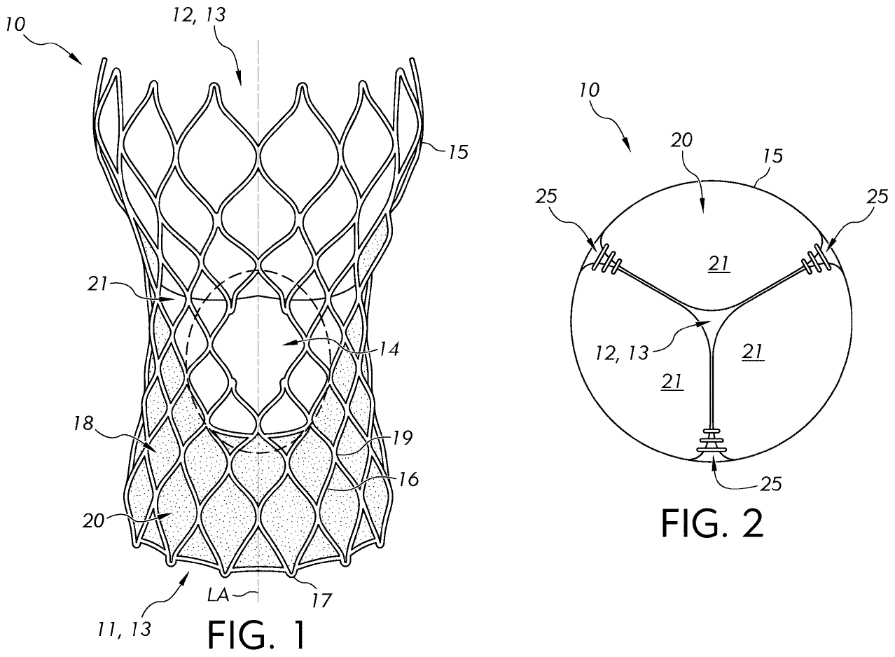

FIGS. 1 and 2 illustrate an example transcatheter heart valve prosthesis 10. The delivery assemblies described herein may be used with the transcatheter heart valve prosthesis 10 and/or other transcatheter heart valve prostheses. The transcatheter heart valve prosthesis 10 is illustrated to facilitate description of the disclosure. The following description of the transcatheter heart valve prosthesis 10 is merely exemplary in nature and is not intended to limit the invention or the application and uses of the invention.

FIGS. 1 and 2 illustrate a side view and a top (outflow end) view, respectively, of the transcatheter heart valve prosthesis 10. The transcatheter heart valve prosthesis 10 includes a radially-expandable frame or stent 15 and a prosthetic valve 20. The frame 15 of the transcatheter heart valve prosthesis 10 supports the prosthetic valve 20 within an interior of the frame 15. In the example transcatheter heart valve prosthesis 10 shown in FIGS. 1 and 2, the frame 15 is self-expandable. However, this is not meant to be limiting, and the frame 15 can be balloon-expandable or mechanically expandable in other embodiments.

The prosthetic valve 20 includes at least one leaflet 21 disposed within and secured to the frame 15. In the embodiment shown in FIGS. 1 and 2, the prosthetic valve 20 includes exactly three leaflets 21, as shown in FIG. 2. However, this is not meant to be limiting, as the prosthetic valve 20 may include more or fewer leaflets 21. The valve leaflets 21 open and close to regulate flow through the transcatheter heart valve prosthesis 10.

As shown in FIG. 1, the transcatheter heart valve prosthesis 10 includes an inflow end 11 and an outflow end 12. The prosthetic leaflets 21 are attached to the frame 15 at commissures 25 such that when pressure at the inflow end 11 exceeds pressure at the outflow end 12, the prosthetic leaflets 21 open to allow blood flow through the heart valve prosthesis 10 from the inflow end 11 to the outflow end 12. When the pressure at the outflow end 12 exceeds pressure at the inflow end 11, the prosthetic leaflets 21 close to prevent blood flow from the outflow end 12 to the inflow end 11. Accordingly, the at least one leaflet (e.g., the prosthetic leaflets 21) can be attached to the plurality of struts 16, for example, by being directly attached to the plurality of struts 16 at the commissures 25, or by being indirectly attached to the plurality of struts 16, for example, by being attached to a skirt, a commissure bracket, or other structure (e.g., mechanical actuator) that is attached to the plurality of struts 16.

The frame 15 of the transcatheter heart valve prosthesis 10 further includes a plurality of struts 16 that are arranged to form a plurality of openings or cells 18 arranged circumferentially around a longitudinal axis LA of the transcatheter heart valve prosthesis 10 and longitudinally to form a tubular structure defining a central lumen 13 of the transcatheter heart valve prosthesis 10. For example, the frame 15 can extend along the longitudinal axis LA between the inflow end 11 and the outflow end 12. The frame 15 is configured to secure the prosthetic valve 20 within the central lumen 13 of the frame 15 and to secure the transcatheter heart valve prosthesis 10 in place in the vasculature of the patient. The struts 16 are defined herein as the elongated wire segments of the frame 15. Struts 16 come together to form crowns 17 or nodes 19, as can be seen in FIG. 1. The frame 15 of the heart valve prosthesis 10 includes a plurality of cells 18 defined as the spaces between the plurality of crowns 17, the plurality of nodes 19, and the plurality of struts 16. The frame 15, and, thus, the plurality of struts 16, can be adjustable between a radially-collapsed position and a radially-expanded position.

In the example embodiment shown in FIG. 1, the plurality of cells 18 may be diamond-shaped. In the example embodiment shown, the plurality of cells include a plurality of first cells 18 and access cells 14. In particular, the access cells are larger than the first cells 18 and can provide access to one or more coronary arteries when the transcatheter heart valve prosthesis 10 is implanted in the patient. In the embodiment shown, there are exactly three access cells 14. However, this is not meant to be limiting, as the frame 15 of the transcatheter heart valve prosthesis 10 can include more, fewer, or no access cells 14. The access cells 14 each have an enlarged area relative or compared to the first cells 18, as can be seen in FIG. 1. Further, the access cells 14 may be located in other locations than the locations shown in FIG. 1. Although not shown, in some embodiments the transcatheter heart valve prosthesis 10 may include an outer skirt extending circumferentially around an outer circumference of the stent 15 at or near the inflow end 11 to prevent paravalvular leakage of blood around the outside of the transcatheter heart valve prosthesis 10 once implanted in the patient.

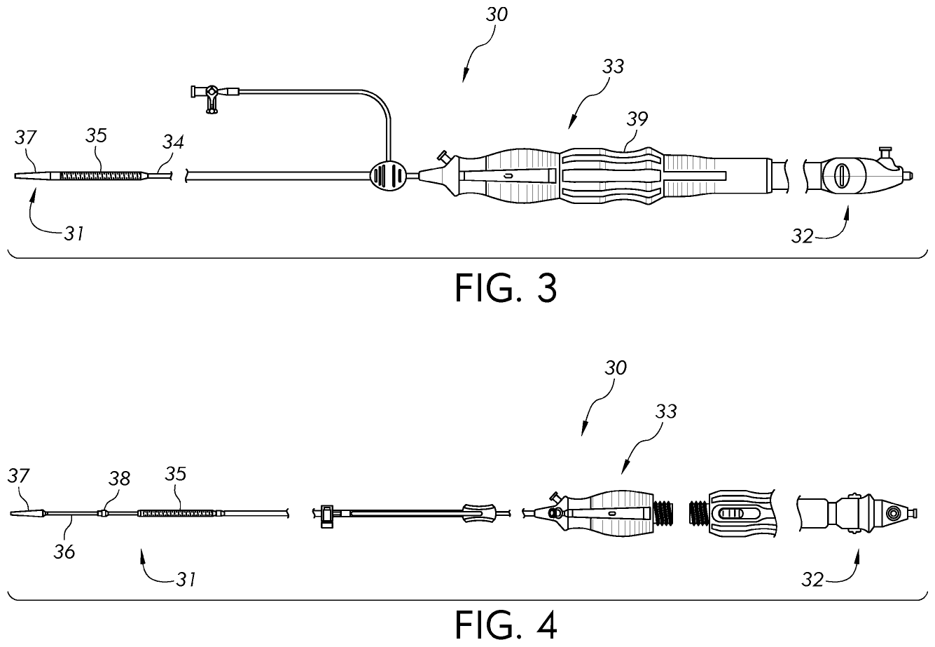

FIGS. 3 and 4 show schematically side views of a delivery assembly 30 for delivering and deploying a transcatheter heart valve prosthesis (e.g., transcatheter heart valve prosthesis 10) according to embodiments hereof. One skilled in the art will realize that FIGS. 3 and 4 illustrate one example of a delivery assembly 30 and that components illustrated in FIGS. 3 and 4 may be removed and/or additional components may be added. The delivery assembly 30 includes a distal end 31, a proximal end 32, and a handle 33. The handle 33 enables a physician to manipulate a distal portion of the delivery assembly 30 and includes actuators for moving parts of the delivery assembly 30 relative to other parts. In the delivery assembly 30, an outer shaft 34 is coupled to an actuator 39 of the handle 33 for moving the outer shaft 34 relative to an inner shaft 36.

A distal portion of the outer shaft 34, referred to as a capsule 35, is configured to surround a transcatheter heart valve prosthesis (e.g., transcatheter heart valve prosthesis 10) during delivery to the treatment site (e.g., a native heart valve) and is retracted from the transcatheter heart valve prosthesis to expose the transcatheter heart valve prosthesis such that it self-expands. The inner shaft 36 is coupled to the handle 33 and movement of the handle 33 translates to movement of the inner shaft 36 and a distal tip or nosecone 37 coupled to a distal end of the inner shaft 36. The inner shaft 36 and distal tip or nosecone 37 may also be translated relative to the outer shaft 34 and the handle 33 via a tip retractor. In the embodiment shown, the inner shaft 36 includes a retainer or spindle 38 for receiving the paddles of the transcatheter heart valve prosthesis 10.

When the actuator 39 is actuated, the actuator 39 moves the outer shaft 34 and the capsule 35 relative to the inner shaft 36, as shown in FIG. 4. As known to those skilled in the art, when the delivery assembly 30 is in position such that the transcatheter heart valve prosthesis 10 is at the desired position at the treatment site in the patient's vasculature, the actuator 39 is actuated to move the capsule 35 relative to the inner shaft 36 and the transcatheter heart valve prosthesis 10 disposed between the inner shaft 36 and the capsule 35, thereby enabling the transcatheter heart valve prosthesis 10 to deploy via self-expansion at the treatment site and release from the retainer 38, as shown in FIG. 4 (without showing the transcatheter heart valve prosthesis 10).

Minimally invasive percutaneous interventional procedures, including endovascular procedures, require access to the venous or arterial system. In general, it is desirable to make the smallest incision point with the shortest tissue contact time when entering the body. Small incisions and short tissue contact time generally lead to improved patient outcomes, less complications, and less trauma to the vessels or organs being accessed, as well as less trauma to the skin and tissue through which the access point is created. Access is required for various medical procedures that deliver or implant structural elements (such as heart valves, heart valve repair devices, occluders, grafts, electrical stimulators, leads, etc.) percutaneously. Some procedures employ relatively large devices that require relatively large sheaths to deliver the devices to the intended site within the body. With such procedures, access site trauma can occur, often resulting in vessel damage, excessive bleeding, increased case time, increased risk of infection, and increased hospitalization time. To reduce access trauma, physicians try to use the smallest devices possible and place the smallest sheath size. This can be problematic, however, if during the procedure the physician discovers a larger device is needed. This leads to a need to upsize the sheath, which is a lengthy procedure and leads to increased risk to the patient. Expandable sheaths can be expanded within the body and thus do not require removal to upsize.

Expandable sheath designs may be regionally or locally expansive to selectively and temporarily expand when the device is passing through a region of the sheath and to retract or recover when the device is not passing or has already passed through the sheath. Embodiments disclosed herein may be employed with an expandable introducer sheath that may solve these and other issues that contribute to vascular trauma. The expandable introducer sheath disclosed herein is described with respect to percutaneous access for transcatheter heart valve repair or replacement, and it should be understood that one or more features of the expandable introducer sheath may be employed alone or in combination for other medical procedures requiring percutaneous access, including but not limited to placement of stents, angioplasty, removal of arterial or venous calcification, and pre-dilatation or post-dilatation.

Various embodiments disclosed herein may include an introducer sheath that has a selectively expandable diameter to allow for the passage of a relatively larger device therethrough and further is configured to return to its original diameter upon passage of the device. The various embodiments may reduce damage to surrounding tissues by reducing contact with those tissues and by eliminating the need to exchange sheaths of different sizes. As a result, in comparison to known sheaths, these embodiments can reduce procedure time, vascular trauma, bleeding, and the resulting risk of infection and other complications. However, it should be understood that the present disclosure is not limited for use with an expandable introducer sheath. Rather, one or more features of the present disclosure can be employed either alone or in combination without an introducer sheath, with a non-expandable introducer sheath, or with an expandable introducer sheath. Likewise, if employed, the introducer sheath may be an integrated introducer sheath (e.g., an introducer sheath integrated with a delivery assembly) or a non-integrated introducer sheath (e.g., an introducer sheath separate from the delivery assembly but provided for use with the delivery assembly).

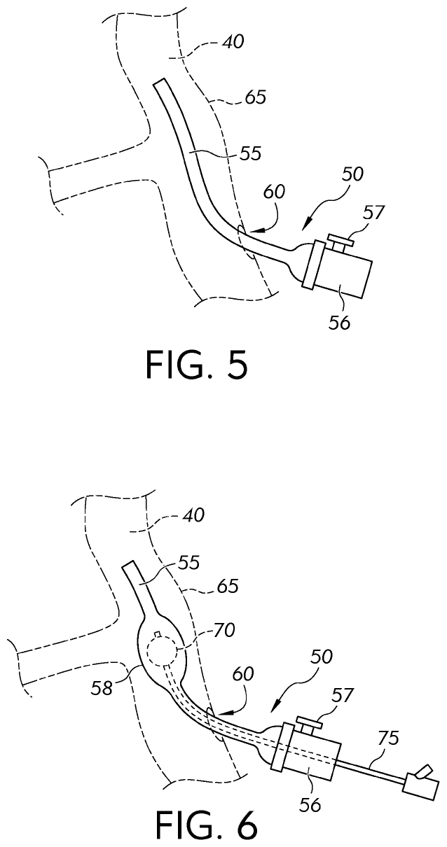

FIGS. 5 and 6 depict one embodiment of an introducer sheath 50 positioned through an incision 60 in the skin 65 of a patient and into a vessel 40 of a patient. The sheath 50 has a tubular shaft 55 and a proximal hub 56 with a hemostatic seal and a luer lock 57. FIG. 5 shows the sheath 50 positioned in the vessel 40 in its normal, unexpanded state, while FIG. 6 shows the sheath 50 positioned in the vessel 40 with a delivery device 75 delivering another device 70 that is being advanced through the sheath 50 such that the tubular shaft 55 expands or deforms at the location where the device 70 is passing through. The shaft 55 expands at expanded region 58 when the device 70 passes through and then retracts or recovers to its original diameter after the device 70 moves past or is removed from the shaft 55. Thus, the tubular shaft 55 is configured to be expandable and retractable.

In certain embodiments, the expandability of the shaft 55 (and any shaft described according to any embodiment set forth herein) is achieved via the elasticity of the shaft 55, which can result in the shaft 55 being either self-expandable or self-expanding or mechanically expandable or mechanically expanding. For purposes of this application, self-expandable means that the shaft 55 is configured to expand to a predetermined or nominal diameter automatically (without any type of actuation, mechanical or otherwise). Further, for purposes of this application, mechanically expandable means that the shaft 55 is configured to expand when a positionable medical device is positioned through the shaft 55. That is, the device itself that is being passed through the shaft 55 causes the expansion of the shaft 55, as depicted in FIG. 6. Alternatively, the expandable characteristics of the shaft 55 can be caused by something other than elasticity.

After passage of the device, the shaft 55 is configured to be contractable, retractable, or recoverable to its original, unexpanded state as depicted in FIG. 5. The retractability can be, in certain embodiments, achieved by the elasticity of the shaft 55, which can result in the shaft 55 being either self-retractable or self-retracting, self-recoverable, or self-contractable, or mechanically retractable or mechanically retracting, mechanically recoverable, or mechanically contractable. For purposes of this application, self-retractable means that the shaft 55 is configured to retract to a predetermined or nominal diameter automatically (without any type of actuation, mechanical or otherwise). Further, for purposes of this application, mechanically retractable means that the shaft 55 is configured to retract when a device or component is used to cause the shaft 55 to retract or recover. Alternatively, the retractable characteristics of the shaft 55 can be caused by something other than elasticity.

For purposes of this application, any device that can be positioned through an introducer sheath according to any embodiment disclosed or contemplated herein can be referred to as a positionable medical device or insertable medical device. Such devices include guidewires, dilators, delivery devices (for delivery and/or placement of structural elements such as heart valves, heart valve repair devices, occluders, grafts, electrical stimulators, leads, etc.), guide catheters, guiding sheaths, diagnostic catheters, stent delivery systems, balloon catheters, and other known vascular devices. Other devices can include non-vascular devices such as scopes and other common surgical instruments. Further, the introducer sheath is configured to receive tissues or organs. Thus, as one non-limiting example, the introducer sheath 50 is described as being an expandable introducer sheath 50 for introduction of a delivery assembly 30 including a transcatheter heart valve prosthesis 10. In further examples, the introducer sheath 50 is employed for introduction of a catheter 851 including an inflation apparatus 801 for pre-dilatation or post-dilatation procedures.

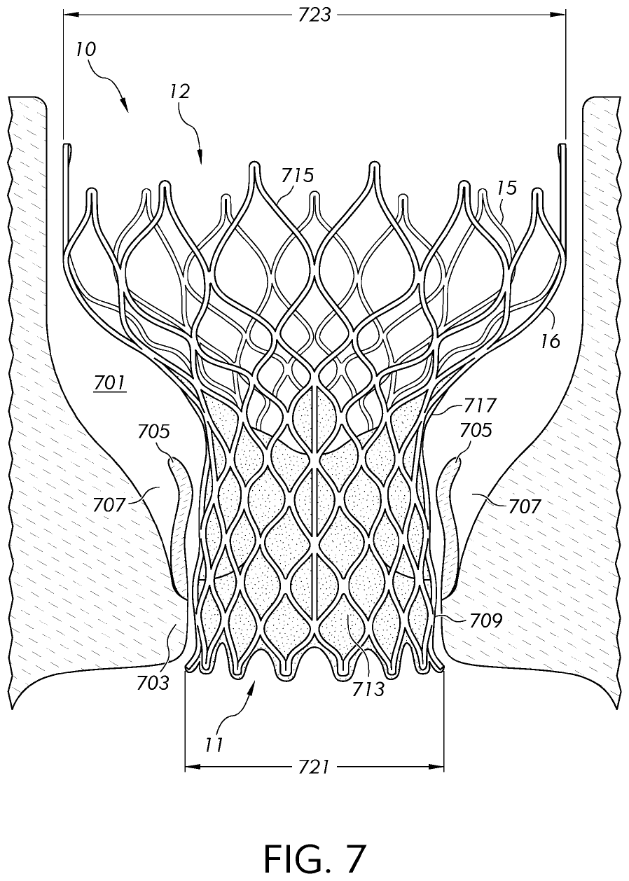

FIG. 7 illustrates the heart valve prosthesis 10 at a treatment site 701 within a patient's vasculature. In aspects, the treatment site 701 can comprise a location of a native aortic annulus (hereinafter “annulus”) 703 of a native heart valve, for example, the annulus of a patient's left ventricle. The treatment site 701 can comprise one or more native valve leaflets 705 and corresponding native sinuses 707. Although disclosed with respect to placement of the heart valve prosthesis 10 within a native anatomy of the patient, in aspects, the heart valve prosthesis 10 can be implanted within a previously implanted prosthetic valve (e.g., a surgical or transcatheter index valve) to facilitate a valve-in-valve (e.g., TAV-in-SAV or TAV-in-TAV) procedure, without departing from the scope of the disclosure. In aspects, paravalvular leakage can occur when blood travels through a gap 709 around the outside of the transcatheter heart valve prosthesis 10, with the gap 709 formed between the transcatheter heart valve prosthesis 10 and the annulus 703. To avoid paravalvular leakage, the heart valve prosthesis 10 can be radially expanded such that an outer radial surface of the heart valve prosthesis 10 can contact the annulus 703 and/or the native valve leaflets 705, thus reducing or eliminating the gap 709 and causing the blood to flow through the central lumen 13 of the heart valve prosthesis 10. The frame 15 of the heart valve prosthesis 10 can comprise an asymmetric hourglass shape with a first section 713 at the inflow end 11, a second section 715 at the outflow end 12, and a waist section 717 positioned between the first section 713 and the second section 715. In aspects, the first section 713 can comprise a first diameter 721 and the second section 715 can comprise a second diameter 723, with the second diameter 723 greater than the first diameter 721. Additionally, as discussed above, in some embodiments the transcatheter heart valve prosthesis 10 may include an outer skirt extending circumferentially around an outer circumference of the prosthesis 15 at or near the inflow end 11 to prevent paravalvular leakage of blood around the outside of the transcatheter heart valve prosthesis 10 once implanted in the patient. Thus, features of the disclosure may be employed alone or in combination with a prosthetic heart valve 10 having an outer skirt or other external sealing member (not shown) or a prosthetic heart valve 10 having no outer skirt.

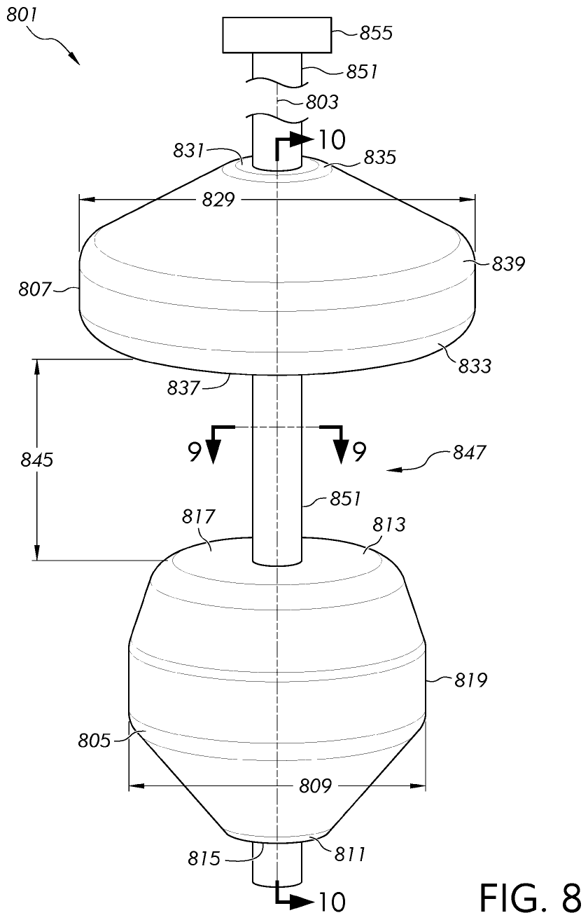

FIG. 8 illustrates a perspective view of an inflation apparatus 801 that can be positioned within the heart valve prosthesis 10 to facilitate radial expansion of the heart valve prosthesis 10. For example, upon being positioned at the treatment site 701, in aspects, the heart valve prosthesis 10 may be partially radially-expanded but not fully radially-expanded, such that the gap 709 may be present. To assist in fully radially-expanding the heart valve prosthesis 10, after the prosthesis 10 is placed at the treatment site 701, the inflation apparatus 801 can be positioned within the central lumen 13 of the heart valve prosthesis 10 to apply an outward radial force to the frame 15 to cause the heart valve prosthesis 10 to fully radially-expand (e.g., post-dilatation).

In addition or alternatively, the inflation apparatus 801 may be employed to apply an outward radial force to a native annulus or to a previously-implanted index valve prior to placement of the heart valve prosthesis 10 to prepare the treatment site 701 for placement of the heart valve prosthesis 10 (e.g., pre-dilatation). The inflation apparatus 801 may be employed to apply an outward radial force to one or more of the frame 15, the annulus 703, and the native leaflets 705, either independently or simultaneously. Outward radial expansion of the inflation apparatus 801 may also loosen, crack, or break calcification that may have accumulated on or around the treatment site 701 (e.g., calcified leaflets 705). By applying force to the calcification deposits, the inflation apparatus 801 may soften (e.g., increase or restore the elasticity of) the treatment site 701, thereby enabling the heart valve prosthesis 10 to expand (e.g., self-expand) more-fully and seal the annulus 703, reducing or eliminating risk of paravalvular leakage. In aspects, the inflation apparatus 801 can be employed pre or post placement of the heart valve prosthesis 10 to alter the shape and/or size of the treatment site 701 as well as the shape and/or size of the heart valve prosthesis 10. For example, in aspects, the inflation apparatus 801 may be employed with a pre-dilatation or a post-dilation procedure to expand a native annulus 701 and/or a heart valve prosthesis 10 from a non-circular (e.g., elliptical) shape to a more circular shape. Without intending to be bound by theory, it is believed that for some patients, a circular annulus may provide better hemodynamic function as compared to the relative hemodynamic function of a non-circular annulus. Outward radial expansion with the inflation apparatus 801 may also increase the effective orifice area (EOA) at the treatment site 701, further improving hemodynamics. In aspects, the inflation apparatus 801 can further be employed to fracture a previously-implanted heart valve prosthesis, for example, prior to implanting a new or redo heart valve prosthesis. For example, in aspects, the inflation apparatus 801 can be employed to fracture a surgical prosthetic index valve and/or a transcatheter prosthetic index valve. Previously-implanted prosthetic heart valves may endothelialize over time rendering the radial stiffness of the previously-implanted prosthetic heart valve stiffer than when the valve was first implanted. By fracturing a previously implanted prosthetic heart valve with the inflation apparatus 801 (e.g., mechanically bending or breaking one or more components of the valve by application of force causing stress), the previously-implanted valve may expand, creating a larger treatment site 701 in which to place the new or redo prosthetic valve and may also decrease in radial stiffness (e.g., become more elastic), enabling better implantation, paravalvular sealing, and hemodynamics of the new or redo prosthetic valve as compared to the relative paravalvular sealing and hemodynamics of a non-fractured, endothelialized prosthetic index valve

The inflation apparatus 801 can extend along an inflation axis 803 and may comprise a first balloon 805 and a second balloon 807. The first balloon 805 and the second balloon 807 are illustrated in an inflated state in FIG. 8, though, the first balloon 805 and the second balloon 807 can be deflated to facilitate movement (e.g., delivery, retraction, etc.) of the inflation apparatus 801 through the patient's vasculature to and from the heart valve prosthesis 10. The first balloon 805 is configured to be positioned in the first section 713 of the heart valve prosthesis 10 and may comprise a first balloon diameter 809. The first balloon 805 can extend along the inflation axis 803 between a first balloon end 811 and a second balloon end 813, with the second balloon end 813 in closer proximity to the second balloon 807 (e.g., with a distance separating the first balloon end 811 and the second balloon 807 greater than the distance separating the second balloon end 813 and the second balloon 807).

The first balloon 805 can comprise one or more walls that can surround and bound an interior chamber of the first balloon 805. For example, the first balloon 805 can comprise a first wall 815, a second wall 817, and an intermediate wall 819 extending between the first wall 815 and the second wall 817. In aspects, the first wall 815 can be positioned at the first balloon end 811 and the second wall 817 can be positioned at the second balloon end 813. In aspects, one or both of the first wall 815 or the second wall 817 may be substantially flat or planar, such that one or both of the first wall 815 or the second wall 817 may be substantially perpendicular to the inflation axis 803. The intermediate wall 819 may extend along, and circumferentially around, the inflation axis 803. In aspects, the intermediate wall 819 may be tapered toward the first wall 815, for example, by comprising a gradually decreasing cross-sectional size toward the first wall 815.

The first wall 815, the second wall 817, and the intermediate wall 819 can be substantially continuous and, in aspects, may be a one-piece formed or composite material. In aspects, the walls 815, 817 819 of the first balloon 805 can comprise a non-compliant material, for example, polyester or nylon. The first balloon 805 can be relatively stiff compared to the stiffness of the second balloon 807 to ensure conformity with a shape of the native annulus 703. In aspects, the first balloon 805 can comprise a variety of sizes based on the cross-sectional size of the native annulus 703. For example, the first balloon diameter 809 can be within a range from about 17 millimeters (“mm”) to about 38 mm, although other diameters less than or greater than this range may also be provided in further aspects.

The second balloon 807 is configured to be positioned in the second section 715 of the heart valve prosthesis 10 and may comprise a second balloon diameter 829. The second balloon 807 can extend along the inflation axis 803 between a first balloon end 831 and a second balloon end 833, with the second balloon end 833 in closer proximity to the first balloon 805 (e.g., with a distance separating the first balloon end 831 and the first balloon 805 greater than the distance separating the second balloon end 833 and the first balloon 805). The second balloon 807 can comprise one or more walls that can surround and bound an interior chamber of the second balloon 807. For example, the second balloon 807 can comprise a first wall 835, a second wall 837, and an intermediate wall 839 extending between the first wall 835 and the second wall 837. In aspects, the first wall 835 can be positioned at the first balloon end 831 and the second wall 837 can be positioned at the second balloon end 833. In aspects, one or both of the first wall 835 or the second wall 837 may be substantially flat or planar, such that one or both of the first wall 835 or the second wall 837 may be substantially perpendicular to the inflation axis 803. The intermediate wall 839 may extend along, and circumferentially around, the inflation axis 803. In aspects, the intermediate wall 839 may be tapered toward the first wall 835, for example, by comprising a gradually decreasing cross-sectional size toward the first wall 835.

The first wall 835, the second wall 837, and the intermediate wall 839 can be substantially continuous and, in aspects, may be a one-piece formed or composite material. In aspects, the walls 835, 837 839 of the second balloon 807 can comprise a semi-compliant material, for example, a high durometer polyurethane material, a polyether block amide, or the like. For example, the second balloon 807 can be relatively less stiff compared to the stiffness of the first balloon 805. In aspects, by providing the second balloon 807 with a relatively less stiff (e.g., more compliant) characteristic compared to the relatively stiffer (e.g., less compliant) characteristic of the first balloon 805, the second balloon 807 can anchor the inflation apparatus 801 within the second section 715 of the prosthetic heart valve 10 without deforming the native anatomy at the second section 715, while the first balloon 805 can radially expand the first section 713 of the prosthetic heart valve 10 and the native anatomy at the first section 713 (e.g., the native annulus 703 and/or the native leaflets 705). Accordingly, in this way, the first balloon 805 and the second balloon 807 may comprise different materials, or, in aspects, the first balloon 805 and the second balloon 807 may comprise the same material. In aspects, the first balloon 805 can comprise a variety of sizes based on the cross-sectional size of the native annulus 703 and/or the second section 715 of the heart valve prosthesis 10. For example, the second balloon diameter 829 can be within a range from about 19 millimeters (“mm”) to about 38 mm. In aspects, the second balloon diameter 829 can be greater than the first balloon diameter 809. In aspects, the catheter 851 can comprise a material that does not radially expand, for example, a polymer such as high-density polyethylene (HDPE), polyethylene high-density, or polyethylene terephthalate.

The inflation apparatus 801 can comprise a catheter 851 extending between the first balloon 805 and the second balloon 807. For example, the inflation apparatus 801, and, thus, the catheter 851, can be a part of the delivery assembly 30 (e.g., illustrated in FIGS. 3-4), for example, with the catheter 851 extending toward the handle 33. In aspects, the catheter 851 can be in operative association with, and, thus, moved and/or controlled by, one or more of the handle 33, the shafts 34, 36, 55, and the actuator 39. In aspects, the inflation apparatus 801 and the catheter 851 can be a separate device from that of the delivery assembly 30. For example, the inflation apparatus 801 and the catheter 851 may correspond to the delivery device 75 and employed with an introducer sheath 50. Thus, the catheter 851 can extend from an exterior of the patient, within the introducer sheath 50, through the incision 60 in the skin 65 (e.g., illustrated in FIGS. 5-6) and into the vessel 40, whereupon the catheter 851 can extend through the first balloon 805, the second balloon 807, and within the gap 847 between the first balloon 805 and the second balloon 807. In aspects, the delivery assembly 30 (FIGS. 3-4) can be employed with the introducer sheath 50 (FIGS. 5-6) to deliver and implant the transcatheter prosthetic heart valve 10. After placement of the transcatheter prosthetic heart valve 10 at the treatment site 701, the delivery assembly 30 can be removed from the patient through introducer sheath 50. The introducer sheath 50 can remain in place within the incision 60 maintaining percutaneous access to the vessel 40. Thus, after removal of the delivery assembly 30 from the introducer sheath 50, the catheter 851 and the inflation apparatus 801 can then be percutaneously introduced into the vessel 40 using the same introducer sheath 50. The inflation apparatus 801 can be employed to provide a post-dilatation of the transcatheter prosthetic heart valve 10.

The inflation apparatus 801 can comprise a fluid source 855 positioned at an exterior of the patient, with the fluid source 855 attached to, and in fluid communication with, the catheter 851. By being in fluid communication, the fluid source 855 can deliver fluid through the catheter 851 to the first balloon 805 and the second balloon 807. In aspects, the fluid delivered by the fluid source 855 can comprise saline mixed with a contrasting agent, though other fluids are envisioned. In aspects, the fluid source 855 and/or the catheter 851 can comprise a flow control device (e.g., a valve, etc.) that can control the flow of the fluid from the fluid source 855 through the catheter 851.

In aspects, the first balloon 805 may be spaced a separating distance 845 from the second balloon 807 to define a gap 847 between the first balloon 805 and the second balloon 807. The separating distance 845 may be measured along the inflation axis 803 between the second balloon end 813 of the first balloon 805 and the second balloon end 833 of the second balloon 807. In aspects, the first balloon 805 can comprise a different shape than the second balloon 807. For example, an axial length (e.g., along the inflation axis 803) of the first balloon 805 can be greater than an axial length of the second balloon 807. In aspects, an axial length of the intermediate wall 819 of the first balloon 805 can be greater than an axial length of the intermediate wall 839 of the second balloon 807. The intermediate wall 819 of the first balloon 805 can extend concentrically about the inflation axis 803 from the second wall 817 toward the first wall 815, before tapering and reducing in cross-sectional size at the first wall 815. The intermediate wall 839 of the second balloon 807 can extend concentrically about the inflation axis 803 from the second wall 837 toward the first wall 835, before tapering and reducing in cross-sectional size at the first wall 835. In aspects, the first walls 815, 835 can be substantially parallel to one another and substantially perpendicular to the inflation axis 803. Likewise, the second walls 817, 837 can be substantially parallel to one another (e.g., and, in aspects, to the first walls 815, 835) and substantially perpendicular to the inflation axis 803. In this way, in aspects, the first balloon 805 can comprise a shape that substantially matches a shape of the first section 713, and the second balloon 807 can comprise a shape that substantially matches a shape of the second section 715.

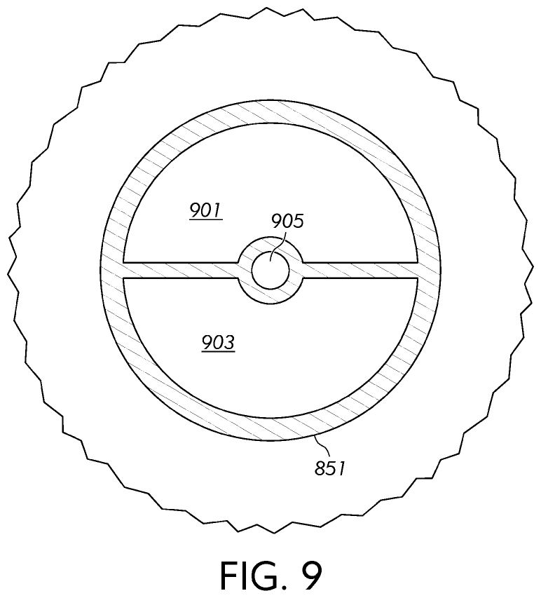

FIG. 9 illustrates a cross-sectional perspective view of the catheter 851 as viewed along lines 9-9 of FIG. 8. In aspects, the catheter 851 can comprise a first hollow chamber 901 in fluid communication with the first balloon 805, a second hollow chamber 903 in fluid communication with the second balloon 807, and a third hollow chamber 905 that can receive a guidewire for guiding the catheter 851. The hollow chambers 901, 903, 905 may extend axially along the length of the catheter 851 such that the fluid source 855 can provide the fluid to the first hollow chamber 901 and the second hollow chamber 903 at an exterior of the patient. In aspects, the first hollow chamber 901 can be positioned on a first side of the catheter 851, and the second hollow chamber 903 can be positioned on an opposing second side of the catheter 851. The third hollow chamber 905 may be located at a center of the catheter 851 and surrounded by the first hollow chamber 901 and the second hollow chamber 903. The catheter 851 can comprise one or more internal walls that can form the hollow chambers 901, 903, 905 such that each hollow chamber is isolated from, and not in fluid communication with, the other hollow chambers.

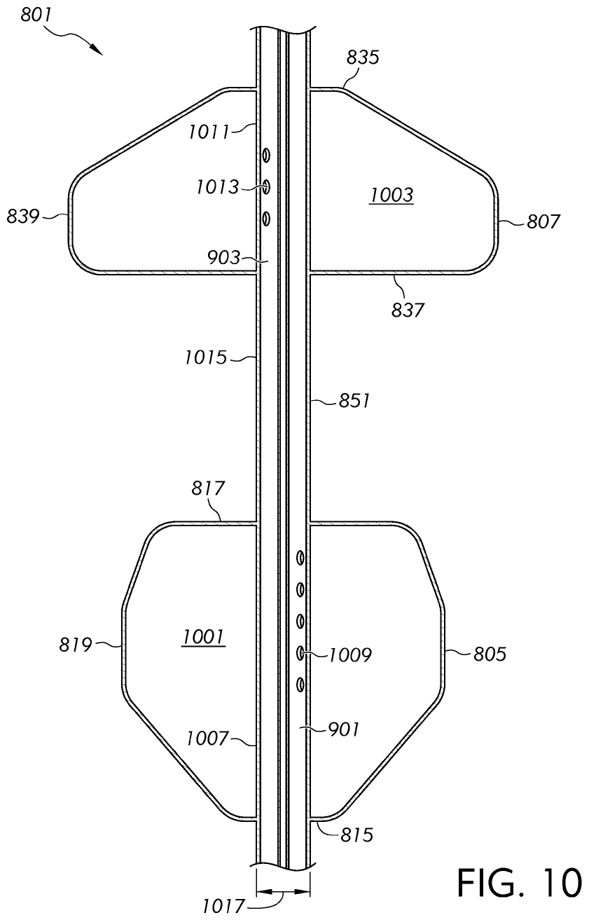

FIG. 10 illustrates a cross-sectional view of the inflation apparatus 801 as viewed along lines 10-10 of FIG. 8, in which an interior of the first balloon 805, the second balloon 807, and the catheter 851 are illustrated. The first balloon 805 can comprise a first interior chamber 1001 surrounded by the walls 815, 817, 819 and the second balloon 807 can comprise a second interior chamber 1003 surrounded by the walls 835, 837, 839. The catheter 851 can comprise a first shaft portion 1007 positioned within, and extending through, the first interior chamber 1001 of the first balloon 805. The first shaft portion 1007 can comprise a first opening 1009, for example, a plurality of first openings 1009, that define a first fluid passageway between the first hollow chamber 901 and the first interior chamber 1001. For example, the first openings 1009 can extend through an outer wall of the catheter 851, with the first openings 1009 in fluid communication with the first hollow chamber 901 of the catheter 851. In aspects, the first shaft portion 1007 may not comprise any openings at the second hollow chamber 903, such that the second hollow chamber 903 is isolated from, and not in fluid communication with, the first interior chamber 1001. When the fluid source 855 delivers fluid to the first hollow chamber 901, the fluid can pass through the first openings 1009 to inflate the first balloon 805. Further, to deflate the first balloon 805, a vacuum can be formed in the first hollow chamber 901 (e.g., via the fluid source 855 or other apparatus) to draw fluid from the first interior chamber 1001, through the first openings 1009, and into the first shaft portion 1007.

The catheter 851 can comprise a second shaft portion 1011 positioned within, and extending through, the second interior chamber 1003 of the second balloon 807. The second shaft portion 1011 can comprise a second opening 1013, for example, a plurality of second openings 1013, that define a second fluid passageway between the second hollow chamber 903 and the second interior chamber 1003. For example, the second openings 1013 can extend through an outer wall of the catheter 851, with the second openings 1013 in fluid communication with the second hollow chamber 903 of the catheter 851. In aspects, the second shaft portion 1011 may not comprise any openings at the first hollow chamber 901, such that the first hollow chamber 901 is isolated from, and not in fluid communication with, the second interior chamber 1003. When the fluid source 855 delivers fluid to the second hollow chamber 903, the fluid can pass through the second openings 1013 to inflate the second balloon 807. Further, to deflate the second balloon 807, a vacuum can be formed in the second hollow chamber 903 (e.g., via the fluid source 855 or other apparatus) to draw fluid from the second interior chamber 1003, through the second openings 1013, and into the second shaft portion 1011.

The catheter 851 can comprise a third shaft portion 1015 that is located between the first shaft portion 1007 and the second shaft portion 1011, with the hollow chambers 901, 903, 905 extending through the shaft portions 1007, 1011, 1015. In aspects, the third shaft portion 1015 may extend between the first balloon 805 and the second balloon 807. The third shaft portion 1015 may comprise zero openings such that the fluid may not exit the catheter 851 through the third shaft portion 1015. In aspects, the catheter 851, for example, the shaft portions 1007, 1011, 1015, can comprise a shaft diameter 1017 that is less than the diameters 809, 829 of the first balloon 805 and the second balloon 807. In aspects, the shaft diameter 1017 may be less than about 4 mm, or less than about 3 mm, or less than about 2 mm. Accordingly, the inflation apparatus 801 can comprise a non-constant diameter along the inflation axis 803 with a larger diameter at the ends (e.g., at the first balloon 805 and the second balloon 807) and a smaller diameter at a center (e.g., at the third shaft portion 1015.

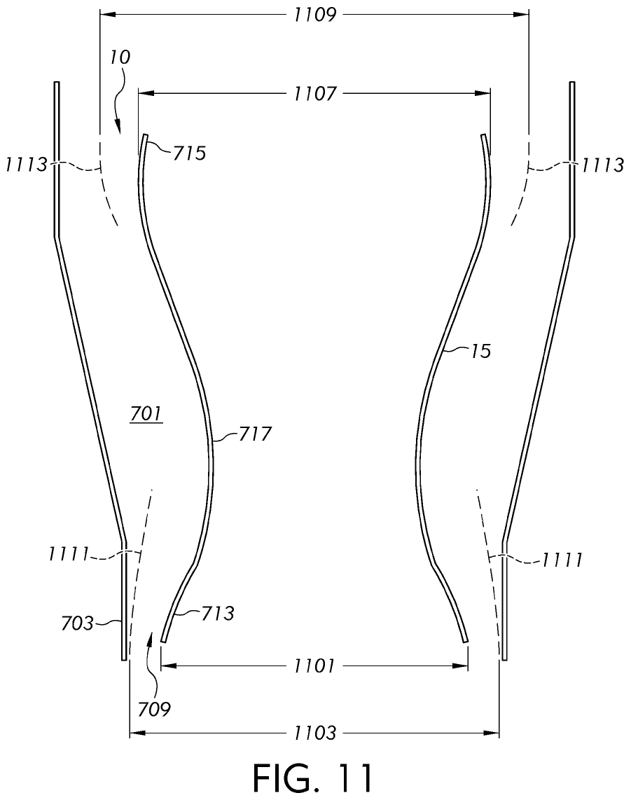

FIG. 11 illustrates a generic/schematic illustration of the heart valve prosthesis 10 positioned at the treatment site 701. For purposes of illustration and to more clearly show the position of the first section 713 relative to the annulus 703, the heart valve prosthesis 10 is illustrated without the struts 16, the valve 20 (e.g., the leaflets 21, commissures 25), etc. However, in operation, the heart valve prosthesis 10 will be similar to the heart valve prosthesis 10 illustrated and described relative to FIGS. 1-10 and 12-16. In aspects, methods of positioning the heart valve prosthesis 10 can comprise moving the heart valve prosthesis 10 to the treatment site 701 within the patient's vasculature in which the heart valve prosthesis 10 is positioned such that the first section 713 is within the annulus 703 at the treatment site 701. The heart valve prosthesis 10 can be moved from a radially-collapsed position to the radially-expanded position. However, in aspects, one or more of the first section 713 or the second section 715 may be under-expanded such that the gap 709 may exist between the native annulus 703 and the first section 713.

In aspects, methods can comprise, prior to positioning the inflation apparatus 801 within the heart valve prosthesis 10, determining that one or more of the first section 713 is radially expanded to a first initial diameter 1101 that is less than a first desired diameter 1103 or the second section 715 is radially expanded to a second initial diameter 1107 that is less than a second desired diameter 1109. For example, with reference to the first section 713, upon being positioned at the treatment site 701, the first section 713 can comprise the first initial diameter 1101 (e.g., measured with respect to the first section 713 with solid lines in FIG. 11). However, the first section 713 may not be fully radially-expanded, such that the gap 709 is present on an outer radial side of the first section 713. The first desired diameter 1103 can correspond to the first section 713 being fully radially-expanded, such that an outer radial surface of the first section 713 may be in contact with the native annulus 703. The first section 713 is illustrated with dashed lines in the fully radially-expanded state, with the dashed lines representing a desired first section position 1111. The desired first section position 1111 is the position of the first section 713 when the first section 713 is radially-expanded to the first desired diameter 1103. In this way, the first desired diameter 1103 is measured with respect to the desired first section position 1111, with the first desired diameter 1103 greater than the first initial diameter 1101.

In addition, or in the alternative, with reference to the second section 715, upon being positioned at the treatment site 701, the second section 715 can comprise the second initial diameter 1107 (e.g., measured with respect to the second section 715 with solid lines in FIG. 11). However, like the first section 713, the second section 715 may not be fully radially-expanded. The second desired diameter 1109 can correspond to the second section 715 being fully radially-expanded. The second section 715 is illustrated with dashed lines in the fully radially-expanded state, with the dashed lines representing a desired second section position 1113. The desired second section position 1113 is the position of the second section 715 when the second section 715 is radially-expanded to the second desired diameter 1109. In this way, the second desired diameter 1109 is measured with respect to the desired second section position 1113, with the second desired diameter 1109 greater than the second initial diameter 1107.

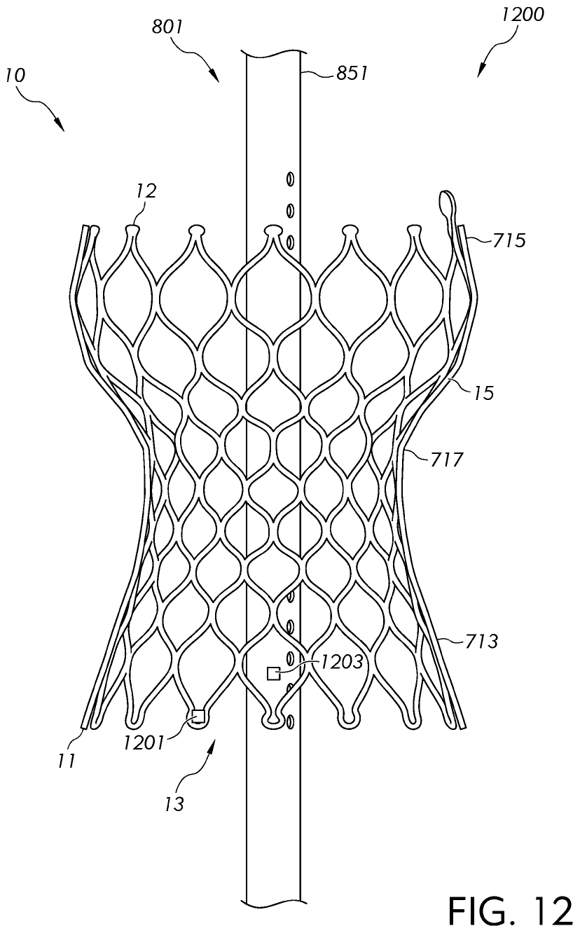

Referring to FIG. 12, to address the heart valve prosthesis 10 being expanded to less than the desired diameter 1103, 1109, methods can comprise positioning the inflation apparatus 801 within the central lumen 13 of the heart valve prosthesis 10. FIG. 12 illustrates a prosthetic heart valve assembly 1200, wherein the prosthetic heart valve assembly 1200 can comprise the heart valve prosthesis 10 and the inflation apparatus 801. For purposes of illustration, the heart valve prosthesis 10 is illustrated without the valve 20 (e.g., the leaflets 21, commissures 25) in FIG. 12 to more clearly show how the inflation apparatus 801 can be positioned within the frame 15. In aspects, the first balloon 805 and the second balloon 807 may be in a deflated state as the inflation apparatus 801 is moved and positioned within the central lumen 13. By being in the deflated state, the first balloon 805 and the second balloon 807 may be flush with the catheter 851 such that the inflation apparatus 801 can occupy a minimum cross-sectional size while moving through the vasculature of the patient.

In aspects, the heart valve prosthesis 10 and the inflation apparatus 801 can comprise radiopaque markers to facilitate alignment. For example, the heart valve prosthesis 10 can comprise a valve radiopaque marker 1201 that, in aspects, may be attached to the frame 15. Though not limited to such a position, in aspects and as illustrated in FIG. 12, the valve radiopaque marker 1201 may be attached to the frame 15 at the inflow end 11. The inflation apparatus 801 can comprise a balloon radiopaque marker 1203 that, in aspects, may be attached to the catheter 851 and positioned within the first balloon 805. The radiopaque markers 1201, 1203 may made of a radiopaque material and/or have echogenic or other properties to be visible from outside the patient's body when using an appropriate imaging technique. The radiopaque markers 1201, 1203 may be made of platinum iridium, tungsten, barium sulfate, other radiopaque materials, and the like. In this way, the radiopaque markers 1201, 1203 can be used to view the movement of the inflation apparatus 801 relative to the heart valve prosthesis 10 and to determine if the inflation apparatus 801 is positioned at a proper location. Accordingly, positioning the inflation apparatus 801 within the central lumen 13 of the heart valve prosthesis 10 can comprise aligning the balloon radiopaque marker 1203 of the inflation apparatus 801 with the valve radiopaque marker 1201 of the heart valve prosthesis 10. By being aligned, the first balloon 805 can be positioned within the first section 713, the second balloon 807 can be positioned within the second section 715, and the third shaft portion 1015 of the catheter 851 can be positioned within the waist section 717. In aspects, aligning the balloon radiopaque marker 1203 can occur prior to one or more of inflating the first balloon 805 or inflating the second balloon 807. In addition, while two radiopaque markers are illustrated in FIG. 12, additional radiopaque markers could be provided to further facilitate positioning of the inflation apparatus 801 relative to the heart valve prosthesis 10, with the additional radiopaque markers attached to various locations on the inflation apparatus 801 and/or the heart valve prosthesis 10.

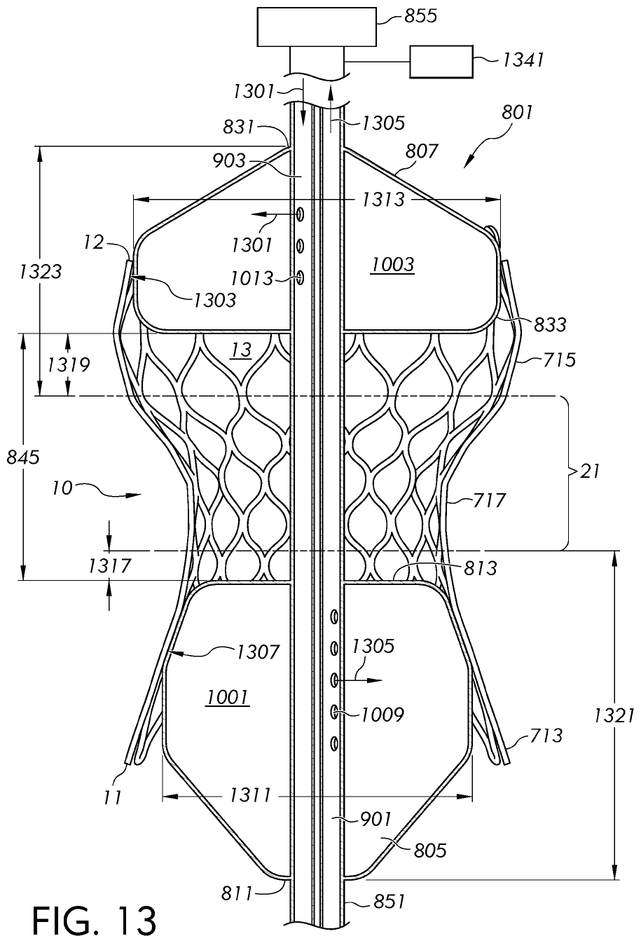

FIG. 13 illustrates a cross-sectional view of the inflation apparatus 801 similar to FIG. 10, but with the inflation apparatus 801 positioned within the central lumen 13 of the heart valve prosthesis 10 and inflated. For example, after the inflation apparatus 801 is aligned with the frame 15 via the radiopaque markers 1201, 1203, the balloons 805, 807 can be inflated. In aspects, the balloons 805, 807 can be inflated non-simultaneously (e.g., with one balloon inflated first followed by inflation of the other balloon) or simultaneously. In aspects, the balloons 805, 807 can be partially inflated non-simultaneously (e.g., with one balloon partially or fully inflated followed by partial or full inflation of the other balloon). In aspects, partial non-simultaneous inflation can be repeated or alternated until the balloons 805, 807 are fully inflated. Additionally or alternatively, the balloons 805, 807 can be partially non-simultaneously inflated and/or deflated such that desired radially expansion of respective balloons 805, 807 can be selectively controlled.

In aspects when the balloons 805, 807 are inflated non-simultaneously, the second balloon 807 can be inflated first. For example, with the second balloon 807 positioned in the second section 715, the second balloon 807 can be pressurized from the deflated state (e.g., illustrated in FIG. 12) to the inflated state. Methods can comprise inflating the second balloon 807 by delivering a second fluid 1301 through the second hollow chamber 903 of the catheter 851 to the second balloon 807 such that the second balloon 807 can contact an interior surface 1303 of the second section 715 to increase the diameter of the second section 715. The second fluid 1301 can be delivered from the fluid source 855, through the second hollow chamber 903, and through the second openings 1013 of the second shaft portion 1011, whereupon the second fluid 1301 can flow into the second interior chamber 1003 of the second balloon 807. In this way, inflating the second balloon 807 can comprise contacting the interior surface 1303 of the second section 715 of the valve prosthesis 10 to radially expand the second section 715 from the second initial diameter 1107 to the second desired diameter 1109. Accordingly, the second balloon 807 can comprise a second balloon diameter 1313 in the inflated state such that the second balloon 807 is in contact with the interior surface 1303 of the second section 715. The second balloon diameter 1313 may be substantially equal to the second desired diameter 1109 of the second section 715.

In aspects, following the inflation of the second balloon 807, the first balloon 805, which is positioned in the first section 713, can be pressurized from the deflated state (e.g., illustrated in FIG. 12) to the inflated state. In this way, inflating the second balloon 807 can occur prior to inflating the first balloon 805. Methods can comprise inflating the first balloon 805 by delivering a first fluid 1305 through the first hollow chamber 901 of the catheter 851 to the first balloon 805 such that the first balloon 805 can contact an interior surface 1307 of the first section 713 to increase the diameter of the first section 713. The first fluid 1305 can be delivered from the fluid source 855, through the first hollow chamber 901, and through the first openings 1009 of the first shaft portion 1007, whereupon the first fluid 1305 can flow into the first interior chamber 1001 of the first balloon 805. In this way, inflating the first balloon 805 can comprise contacting the interior surface 1307 of the first section 713 of the valve prosthesis 10 to radially expand the first section 713 from the first initial diameter 1101 to the first desired diameter 1103. Accordingly, the first balloon 805 can comprise a first balloon diameter 1311 in the inflated state such that the first balloon 805 is in contact with the interior surface 1307 of the first section 713. The first balloon diameter 1311 may be substantially equal to the first desired diameter 1103 of the first section 713. In aspects, the delivery of the fluids 1301, 1305 can be controlled by a valve, stopcock, or other flow control device 1341 (e.g., illustrated generically in FIG. 13), such that, when desired, one fluid can be selectively and independently delivered at a time to one of the balloons 805, 807.

As discussed above, in aspects, by providing the second balloon 807 with a relatively less stiff (e.g., more compliant) characteristic compared to the relatively stiffer (e.g., less compliant) characteristic of the first balloon 805, the second balloon 807 can be inflated first (e.g., before inflation of the first balloon 805) to anchor the inflation apparatus 801 within the second section 715 of the prosthetic heart valve 10 without deforming the native anatomy at the second section 715. With the inflation apparatus 801 anchored in the second section 715 based on inflation of the second balloon 807, migration or other movement of the inflation apparatus 801 can be reduced or prevented. With migration of the inflation apparatus 801 restricted, the first balloon 805 can then be inflated (e.g., after inflation of the second balloon 807) to radially expand the first section 713 of the prosthetic heart valve 10 and the native anatomy at the first section 713 (e.g., the native annulus 703 and/or the native leaflets 705). Without intending to be bound by theory, it is believed that, in aspects, inflation of the first balloon 805 alone or prior to inflation of the second balloon 807 may be less predictable and less controlled as, without first inflating the second balloon 807 to anchor the inflation apparatus 801 at the treatment site 701, the inflation apparatus 801 and/or the prosthetic heart valve 10 may migrate (e.g., move axially relative to the native annulus 703) such that radial expansion of the first balloon 805 could undesirably occur at an unintended lateral location within the prosthetic heart valve 10 (e.g., at waist section 717), applying an unintentional radial force to, for example, the prosthetic leaflets 21 and/or cause dislodgement or unwanted positioning of the prosthetic heart valve 10 relative to the native annulus 703.

Following the inflation of the balloons 805, 807, the first balloon 805 is inflated and positioned in the first section 713, the second balloon 807 is inflated and positioned in the second section 715, and the catheter 851 can extend through the waist section 717 between the first balloon 805 and the second balloon 807. Following inflation, the first balloon diameter 1311 can be substantially equal to a maximum first diameter of the first section 713, with the maximum first diameter of the first section 713 being substantially equal to the first desired diameter 1103. Likewise, after inflation, the second balloon diameter 1313 can be substantially equal to a maximum second diameter of the second section 715, with the maximum second diameter of the second section 715 being substantially equal to the second desired diameter 1109. In this way, the balloons 805, 807 can facilitate radial expansion of the heart valve prosthesis 10, for example, when the heart valve prosthesis 10 is initially not fully expanded.

Following inflation, the first balloon 805 is spaced the separating distance 845 from the second balloon 807 such that the first interior chamber 1001 is isolated from the second interior chamber 1003. By being isolated, fluid may not flow between the first interior chamber 1001 and the second interior chamber 1003 when the balloons 805, 807 are inflated. As such, the balloons 805, 807 may not contact the leaflets 21 (e.g., illustrated schematically in FIG. 13 with dashed lines to illustrate a position of the leaflets 21 within the frame 15). For example, the first balloon 805 can be spaced a first distance 1317 from the plurality of leaflets 21 and the second balloon 807 can be spaced a second distance 1319 from the plurality of leaflets 21. For example, the first balloon 805 can extend between the first balloon end 811 and the second balloon end 813, with the first balloon end 811 spaced a distance 1321 from the plurality of leaflets 21 and the second balloon end 813 spaced the first distance 1317 from the plurality of leaflets 21. The second balloon 807 can extend between the first balloon end 831 and the second balloon end 833, with the first balloon end 831 spaced a distance 1323 from the plurality of leaflets 21 and the second balloon end 833 spaced the second distance 1319 from the plurality of leaflets 21. Accordingly, by being spaced apart and not in contact with the leaflets 21, the first balloon 805 and the second balloon 807 may not interfere with the operation of the leaflets 21 by causing unwanted radial expansion of the leaflets 21.

Following the radial expansion of the heart valve prosthesis 10 by the inflation apparatus 801, the balloons 805, 807 can be deflated and the inflation apparatus 801 can be removed from the central lumen 13 of the heart valve prosthesis 10. To deflate the balloons 805, 807, a vacuum can be formed within the hollow chambers 901, 903 of the catheter 851, thus drawing fluid from the balloons 805, 807 and into the catheter 851. In this way, fluid can continue to be removed from the balloons 805, 807 until the balloons 805, 807 are in a fully deflated state (e.g., illustrated in FIG. 12). Upon deflating the balloons 805, 807, the inflation apparatus 801 can be retracted and withdrawn from the heart valve prosthesis 10, and removed from the patient's vasculature.

In aspects, the inflation apparatus 801 is not limited to comprising both the first balloon 805 and the second balloon 807. Rather, in aspects, the inflation apparatus 801 may comprise a single balloon. For example, the inflation apparatus 801 may comprise the first balloon 805 and not the second balloon 807. In such an embodiment, the first balloon 805 can function to radially-expand the first section 713 to the first desired diameter 1103. This radial expansion of the first section 713 can reduce or eliminate the gap 709, thus reducing the likelihood of paravalvular leakage. Further, by radially expanding the first section 713 with the first balloon 805, the other portions of the heart valve prosthesis 10 may at least partially radially expand as well due to the proximity to the first section 713.

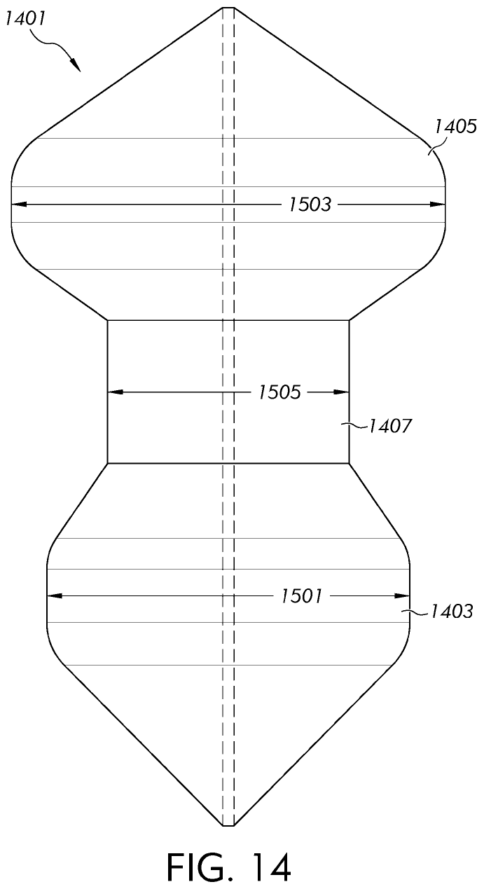

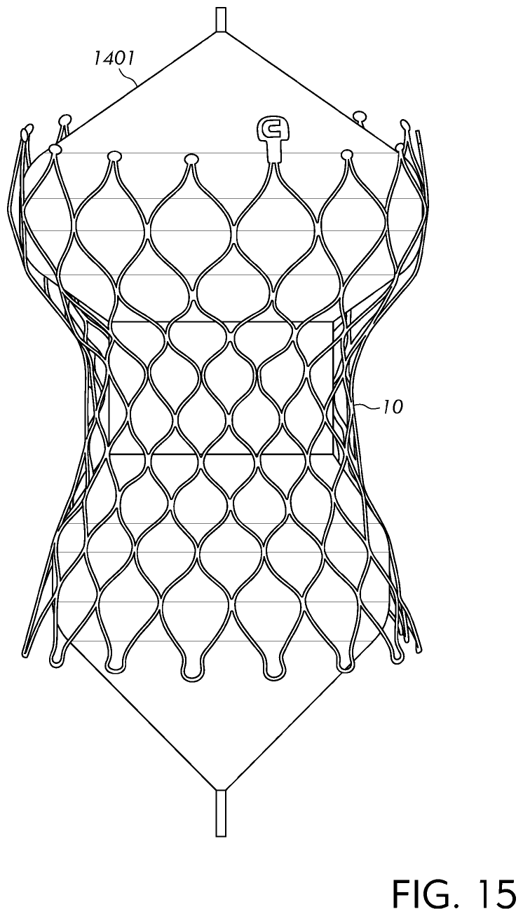

FIGS. 14-15 illustrates additional embodiments of an inflation apparatus 1401 that can be used to radially-expand the heart valve prosthesis 10 in a substantially identical manner as described above relative to FIGS. 7-13. For example, in aspects, the inflation apparatus 1401 can comprise a first balloon 1403, a second balloon 1405, and a central balloon 1407. The first balloon 1403 can be positioned in the first section 713, the second balloon 1405 can be positioned in the second section 715, and the central balloon 1407 can be positioned in the waist section 717. In aspects, the first balloon 1403, the second balloon 1405, and the central balloon 1407 can comprise a single, continuous interior chamber such that the balloons 1403, 1405, 1407 are in fluid communication with one another. The central balloon 1407 can be positioned in the waist section 717 and may be attached at one end to the first balloon 1403 and at an opposing end to the second balloon 1405. The catheter 851 can extend into an interior chamber of the balloons 1403, 1405, 1407 to deliver the fluid from the fluid source 855 to the interior chamber, thus causing the balloons 1403, 1405, 1407 to inflate.

FIG. 15 illustrates the inflation apparatus 1401 positioned within the heart valve prosthesis 10. The first balloon 1403 can comprise a first balloon diameter 1501, the second balloon 1405 can comprise a second balloon diameter 1503, and the central balloon 1407 can comprise a central balloon diameter 1505. The central balloon diameter 1505 may be less than the first balloon diameter 1501 and the second balloon diameter 1503. In aspects, the first balloon diameter 1501 may be substantially equal to the first desired diameter 1103 of the first section 713 and the second balloon diameter 1503 may be substantially equal to the second desired diameter 1109 of the second section 715. The central balloon diameter 1505 may be small enough such that the central balloon 1407 may not contact the leaflets 21 or cause the leaflets 21 to radially expand. Accordingly, several different types of inflation apparatuses 801, 1401 are envisioned that can assist in radially-expanding the heart valve prosthesis 10 to reduce the likelihood of paravalvular leakage. Further, due to the non-constant cross-sectional size of the inflation apparatuses 801, 1401 (e.g., with the ends comprising a larger diameter than the center), the inflation apparatuses 801, 1401 will not damage or interfere with the leaflets 21 and, also, will not damage the native structures at the treatment site 701.

It should be understood that while various aspects have been described in detail relative to certain illustrative and specific examples thereof, the present disclosure should not be considered limited to such, as numerous modifications and combinations of the disclosed features are possible without departing from the scope of the following claims.

What is claimed is:

1. An inflation apparatus for dilating a prosthetic heart valve comprising an annular frame extending along a longitudinal axis between an inflow end and an outflow end, the frame comprising a plurality of struts and configured to be adjustable between a radially-collapsed position and a radially-expanded position, the frame comprising a first section at the inflow end, a second section at the outflow end, a waist section positioned between the first section and the second section, and a valve prosthesis comprising at least one leaflet attached to the plurality of struts; the inflation apparatus comprising:
a first balloon configured to be positioned in the first section, the first balloon configured to be pressurized from a deflated state to an inflated state, the first balloon comprising a first balloon diameter in the inflated state such that the first balloon is configured to be in contact with an interior surface of the first section;
a second balloon configured to be positioned in the second section, the second balloon configured to be pressurized from a deflated state to an inflated state, the second balloon comprising a second balloon diameter in the inflated state such that the second balloon is configured to be in contact with an interior surface of the second section; and
a catheter configured to extend through the waist section between the first balloon and the second balloon, the catheter comprising a first hollow chamber in fluid communication with the first balloon and a second hollow chamber in fluid communication with the second balloon.

2. The inflation apparatus of claim 1, wherein the first balloon is spaced a separating distance from the second balloon and defining a gap between the first balloon and the second balloon, the catheter extending within the gap.

3. The inflation apparatus of claim 2, wherein the first balloon is configured to be spaced a first distance from the at least one leaflet and the second balloon is configured to be spaced a second distance from the at least one leaflet.

4. The inflation apparatus of claim 3, wherein the first balloon diameter is substantially equal to a maximum first diameter of the first section.

5. The inflation apparatus of claim 4, wherein the second balloon diameter is substantially equal to a maximum second diameter of the second section.

6. The inflation apparatus of claim 1, wherein the catheter comprises a first shaft portion positioned within the first balloon, the first shaft portion comprising a first opening defining a first fluid passageway between the first hollow chamber and a first interior chamber of the first balloon.

7. The inflation apparatus of claim 1, wherein the catheter comprises a second shaft portion positioned within the second balloon, the second shaft portion comprising a second opening defining a second fluid passageway between the second hollow chamber and a second interior chamber of the second balloon.

8. An inflation apparatus for dilating a prosthetic heart valve comprising an annular frame extending along a longitudinal axis between an inflow end and an outflow end, the frame comprising a plurality of struts and configured to be adjustable between a radially-collapsed position and a radially-expanded position, the frame comprising a first section at the inflow end, a second section at the outflow end, a waist section positioned between the first section and the second section, and a valve prosthesis comprising at least one leaflet attached to the plurality of struts; the inflation apparatus configured to be positing within the frame and comprising:
a first balloon configured to be positioned in the first section, the first balloon configured to be pressurized from a deflated state to an inflated state, the first balloon comprising a first balloon diameter in the inflated state such that the first balloon is configured to be in contact with an interior surface of the first section, the first balloon extending between a first balloon end and a second balloon end, the first balloon end configured to be spaced a first distance from the at least one leaflet and the second balloon end configured to be spaced a second distance from the at least one leaflet;
a catheter configured to extend through the interior of the frame and comprising a first hollow chamber in fluid communication with the first balloon; and
a fluid source in fluid communication with the catheter, the fluid source configured to deliver a fluid through the first hollow chamber to the first balloon.

9. The inflation apparatus of claim 8, further comprising:
a second balloon configured to be positioned in the second section, the second balloon configured to be pressurized from a deflated state to an inflated state, the second balloon comprising a second balloon diameter in the inflated state such that the second balloon is configured to be in contact with an interior surface of the second section; and
a central balloon configured to be positioned in the waist section and attached at one end to the first balloon and at an opposing end to the second balloon, the first balloon, the second balloon, and the central balloon defining a continuous interior chamber.

10. The inflation apparatus of claim 9, wherein the central balloon comprises a central balloon diameter that is less than the first balloon diameter and the second balloon diameter.

11. The inflation apparatus of claim 10, wherein the catheter extends through the first balloon, the second balloon, and the central balloon, the catheter comprising one or more openings defining a fluid passageway between the first hollow chamber and the interior chamber.
